(19) United States
(12) Patent Application Publication
(10) Pub. No.: US 2002/0032430 A1
(43) Pub. Date: Mar. 14, 2002

(54) Method and system for treating stroke using hypothermia

(76) Inventors: Xia Luo, Los Angeles, CA (US); Scott M. Evans, Santa Ana, CA (US); William J. Worthen, Coto de Caza, CA (US)

Correspondence Address: ALSIUS CORPORATION, Suite 150, 15770 Laguna Canyon Road, Irvine, CA, 92618, US

(21) Appl. No.: 09/900,378

(22) Filed: Jul. 6, 2001

Related U.S. Application Data
(62) Division of application No. 09/456,110, filed on Dec. 7, 1999.

(51) Int. Cl. A61M 31/00

(52) U.S. Cl. 604/512

(57) ABSTRACT

A method for treating stroke patients includes inducing mild or moderate hypothermia in a patient using one or more closed loop heat exchange catheters positioned either in the patient's central venous system, carotid artery or both when the patient's body temperature and/or brain temperature are above predetermined thresholds. Additional steps for treating and managing a stroke patient can also be undertaken, e.g., infusing paralytics, infusing neuro-protectants, infusing anti-clot and/or clot lysis medications, performing neuro-protection procedures, performing non-catheter-based hypothermia, performing angioplasty, deploying stent, removing clot(s), maintaining a predetermined ICP level such as draining CSF, and/or ventilating the patient.

### FIELD OF INVENTION

The present invention relates to methods and systems for managing stroke conditions.

### BACKGROUND

Every year, approximately 700,000 Americans suffer a stroke. It is estimated that about 30% of stroke victims die within 3 months, and a significant number of those who survive need assistance in their daily routine. Stroke is considered the third leading killer in the United States, following cancer and heart disease.

There are two types of strokes: ischemic and hemorrhagic. Ischemic stroke is caused by a blockage in the artery, thereby cutting off the oxygen supply to the brain cells. Hemorrhagic stroke is caused by an intracerebral hemorrhage or a subarachnoid hemorrhage. Studies have shown that an association exists between body temperature and initial stroke severity, infarct size, mortality and outcome in survivors. For example, it was reported that a difference in body temperature of 1 degree C was equivalent to a 4-point difference in Scandinavian Stroke Scale (“SSS”) on hospital admission, a 15 mm difference in infarct size, an 80% difference in mortality, and a 4-point difference in SSS score at discharge (Reith et al., Body Temperature in Acute Stroke; Relation to Stroke Severity, Infarct size, Mortality, and Outcome, The Lancet, Vol. 347, pp. 422-425, Feb. 17, 1996). Thus, if body temperature can be reduced quickly in a stroke patient, significant improvements may be achieved in reducing mortality and outcome in the survivors.

Accordingly, studies suggest that cooling the stroke patient below the normal body temperature (˜37° C.) is preferable. In particular, the stroke patient's condition may be significantly improved if the patient's body temperature is mildly or moderately cooled to about 32° C.-36° C. relatively quickly for a short period, e.g., 1-2 hours. And if desirable, the patient's body temperature can be maintain at about about 32° C.-36° C. for approximately 12-72 hours. The typical methods of cooling patient body temperature are externally applied, including the use of cooling blankets, ice packs, cooled air fans, immersion in ice water, enemas and ravages. Often, these methods are difficult to control, cumbersome and labor intensive (Gobin et al., Heat Exchange Catheter for Therapeutic Hypothermia and Temperature Control, Congress of Neurological Surgeons 49^{th} Annual Meeting, October-November 1999, Abstract No. 101). Accordingly, it would be advantageous to provide a systematic cooling method that is effective, is easy to use and requires minimal added work for medical personnel.

Systems and methods have been disclosed that propose cooling blood which flows to the brain through the carotid artery. An example of such systems and methods is disclosed in co-pending U.S. patent application Ser. No. 09/063,984, filed Apr. 21, 1998, owned by the present assignee and incorporated herein by reference. In the referenced application, various catheters are disclosed which can be advanced into a patient's carotid artery and through which coolant can be pumped in a closed circuit to remove heat from the blood in the carotid artery and thereby cool the brain. The referenced devices have the advantage over other methods of cooling (e.g., wrapping patients in cold blankets, etc.) of being controllable, of being relatively easy to use, and of being capable of rapidly cooling and maintaining blood temperature at a desired set point.

As recognized in co-pending U.S. patent application Ser. No. 09/133,813, filed Aug. 13, 1998, owned by the present assignee and incorporated herein by reference, the above-mentioned advantages in treating stroke patients by internal cooling can also be realized by cooling the patient's entire body, i.e., by systemic, internally-induced hypothermia. The advantage of systemic hypothermia is that, as recognized by the present assignee, to induce systemic hypothermia a cooling catheter or other cooling device need not be advanced into the blood supply of the brain, but rather can be easily and quickly placed into the relatively large vena cava of the central venous system. Moreover, since many patients already are intubated with central venous catheters for other clinically approved purposes, providing a central venous catheter that can also cool the blood requires no additional surgical procedures for those patients. A cooling central venous catheter is disclosed in the present assignee's co-pending U.S. patent application Ser. No. 09/253,109, filed Feb. 19, 1999 and Ser. No. 09/305,613, filed May 5, 1999, both of which are incorporated herein by reference.

### SUMMARY OF THE INVENTION

The present invention relates to methods and systems for treating stroke patients who can benefit from hypothermia treatment. In one embodiment, the method for treating a stroke condition includes identifying that the patient has had a stroke, and in response, lowering the patient's temperature using at least one heat exchange catheter placed in the central venous system of the patient. In another embodiment, a heat exchange catheter is placed through the carotid artery of the patient to lower the temperature of the patient's brain. In yet another embodiment, the patient may receive at least two heat exchange catheters, one placed in the central venous system, and another placed through the carotid artery to lower the body temperature with directed focus on the brain. In this embodiment, the heat exchange rates of each heat exchange catheter may be adjusted separately depending on the patient's relative body and brain temperatures.

In one embodiment, the method includes determining the blood pressure and the blood volume of the patient, and maintaining the blood pressure and blood volume at the desired levels determined by the patient's caregiver. In one embodiment, the patient is given sedative medication. The sedative medication can be introduced into the patient's blood stream through the medicine dispensing port of the heat exchange catheter. In some cases, it may be desirable to intubate and maintain the patient on a ventilator to increase the amount of air entering the patient's pulmonary system. Further, the patient may be given neuro-protectant medication to prevent further damage to the cerebral tissues. In one embodiment, the neuro-protectant medication is introduced into the patient's blood stream through the medicine dispensing port of the heat exchange catheter. For some patients, a paralytic drug may be administered to curb shivering episodes. Again, the paralytic medication may be given to the patient through the medicine dispensing port of the heat exchange catheter. It will be apparent to those skilled in the art that the medicine dispensing port of the heat exchange catheter can dispense various other medications deemed appropriate for each patient.

For some patients, there may be a need to perform additional neuro-protection procedures such as but not limited to bypass surgery and perfusion procedures to deliver oxygenated blood to the brain cells. Additionally, patients may receive non-catheter-based hypothermia treatment which may include, but is not limited to, applying cooling blankets and/or ice packs on the patient, blowing cold air fans on the patient or immersing the patient in a tub of cold, icy water. Moreover, the method may include monitoring and maintaining the patient's intracranial pressure (ICP) at a level appropriate for the patient's medical condition.

In one embodiment directed to the treatment of ischemic stroke patients, the method may include administering anti-clot drugs and or clot lysis drugs. Again, the anti-clot and clot lysis drugs may be dispensed through the medicine dispensing port of the heat exchange catheter. In another embodiment, the method may include performing an angioplasty procedure on one or more arteries associated with the stroke to eliminate the areas of narrowing in the blood vessels. In another embodiment, a stent is deployed into one or more arteries to provide structural support in the enlarged flow areas of the blood vessels. It will be understood by those skilled in the art which arteries are candidates for angioplasty or stent procedures. In many ischemic stroke patients, the angioplasty and stent procedures are performed on the carotid artery.

Further, any remaining clots to the blood vessels can be cleared away through removing the clot(s) by the application of laser, ultrasonic energies or mechanical device at the blockage areas, or by surgically removing the clots.

In another aspect, a system for treating a stroke patient includes at least one cooling catheter having a heat exchange region on its distal portion and at least one blood pressure probe to provide an indication of said patient's blood pressure.

It is the object of the present invention to provide a systematic cooling method and system that are effective, are easy to use and require minimal added work for medical personnel. Additional objects and advantages of the invention will be set forth in part in the description which follows, and may be obvious from the description or learned by practice of the invention. The objects and advantages of the invention also may be realized and attained by means of the method acts, instrumentalities and combinations particularly pointed out in the appended claims.

### BRIEF DESCRIPTION OF THE DRAWING

The details of the present invention, both as to its structure and operation, can best be understood in reference to the accompanying drawings, in which like reference numerals refer to like parts, and to the accompanying claims. The accompanying drawings, which are incorporated in and constitute a part of the specification, illustrate preferred embodiments of the invention, and together with the description serve to explain the principles of the invention.

FIG. 1 is a schematic view of the cooling system using the first cooling catheter.

FIG. 2 is a schematic view of the cooling system using the second cooling catheter.

FIG. 3 is a flow chart of one embodiment of the present invention for treating a stroke patient.

FIG. 4 is a flow chart of one embodiment of the present invention for treating an ischemic stroke patient.

### DETAILED DESCRIPTION OF THE PREFERRED EMBODIMENT

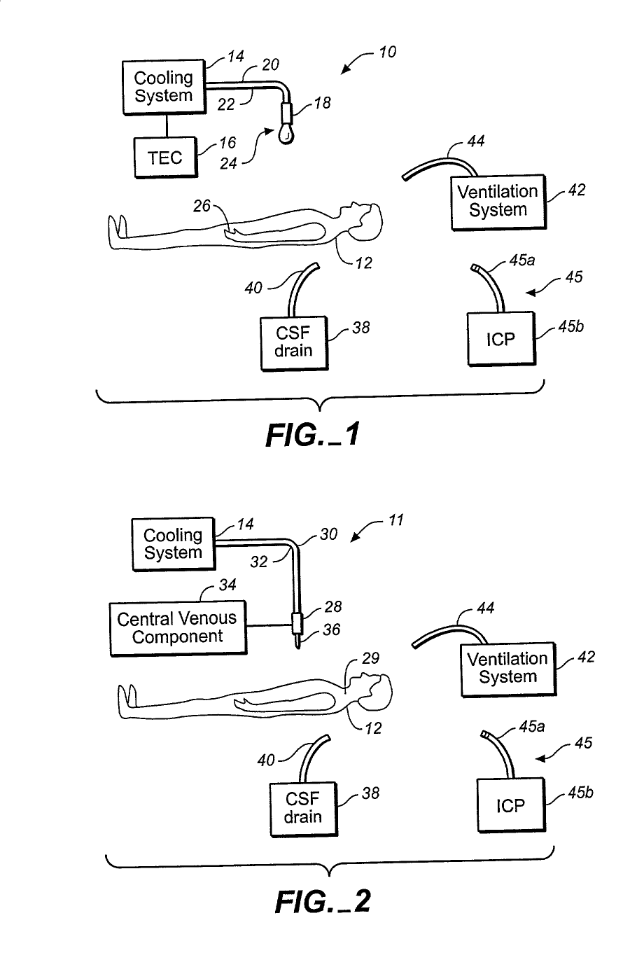

Referring to FIG. 1, a therapeutic system 10 is shown for treating a stroke patient 12. As shown, the system 10 includes a cooling system 14 that can be a water-bath system such as the system disclosed in the present assignee's co-pending U.S. patent application Ser. No. 09/220,897 filed Dec. 28, 1998 and incorporated herein by reference, or a cooling system including at least one thermal electric cooler (TEC) 16, as disclosed in the present assignee's co-pending U.S. patent application Ser. No. 09/260,950, filed Mar. 2, 1999 and incorporated herein by reference. In any case, the cooling system 14 can be considered a source of coolant, preferably sterile saline for the catheters of the present invention.

As set forth in these applications, the cooling system 14 can include a heat exchanger, a pump and if desired, a controller. Preferably, the pump is a peristaltic pump, but other types of positive displacement pumps such as, but not limited to, piston pumps and gear pumps, or even centrifugal pumps, can be used. A peristaltic pump is preferred in the present implementation because it can pump coolant without directly contacting the coolant, but instead simply by squeezing a tube through which coolant flows. In this way, the pump is reusable, and only the present catheters and portions of the system 10 coming in direct contact with the coolant need be made disposable to render an advantageously disposable and sterile coolant delivery system. The controller controls the rate at which coolant is pumped and, if desired, the rate at which heat is added or subtracted from the coolant. The controller can be implemented by a software-executing processor or by discrete logic circuits or other electronic circuitry to establish a desired patient temperature by appropriately controlling the pump and/or heat exchanger in response to a temperature signal derived from a sensor in the patient 12. Other implementations of the controller will be understood by one skilled in the art.

As shown in FIG. 1, a first cooling catheter 18 can communicate with the cooling system 14 via coolant supply and return lines 20, 22. The coolant lines 20, 22 can be IV lines or tubes or other suitable fluid conduits, such as metal (steel) tubes. When the coolant lines 20, 22 are plastic tubes, they can be connected to the catheter 18 and the cooling system 14 by suitable connecting structures, such as Luer fittings, interference fits, solvent bonding, heat staking, ultrasonic welding and the like.

The first cooling catheter 18 includes a heat exchange region 24. The heat exchange region 24 can be established by one or more hollow fibers, as disclosed in the above-referenced U.S. patent application Ser. No. 09/133,813. Alternatively, the heat exchange region 24 can include one or more cooling membranes such as balloons as disclosed in the above-referenced U.S. patent application Ser. Nos. 09/253,109 and 09/305,613. For example, the heat exchange region 24 of the first catheter 18 can be established by one to ten axially staggered balloons, each balloon being two to fifteen millimeters in diameter when inflated with coolant.

As set forth in the referenced applications, coolant fluid is circulated in a closed fluid communication loop between the heat exchanger region 24 and the cooling system 14 to remove heat from the patient 12. In another aspect, the coolant fluid temperature can be adjusted through the controller to provide heated fluid (rather than cooled fluid) to the patient's blood stream whereby the body temperature of the patient increases. In this aspect, the heated fluid is circulated in a closed fluid communication loop between the heat exchanger region 24 and the system 14 to add heat to the patient 12.

As set forth in greater detail below, the first catheter 18 is advanced (for example, through an introducer sheath or a guidewire) into the vena cava of the patient 12 through a groin entry point 26 to establish hypothermia in the patient 12. Preferably, the catheter 18 is advanced either through the saphenous, femoral or iliac vein. Additionally, the first catheter 18 can be advanced into the carotid artery through a neck entry point 27 (not shown) to establish hypothermia in the patient 12.

Referring to FIG. 2, a second therapeutic system 11 is shown for treating a stroke patient 12. In addition or in lieu of the first catheter 18, a second cooling catheter 28 which is configured for use as a central venous catheter can be advanced into the central venous system of the patient 12 through a neck entry point 29. The second catheter 28 can be embodied by the catheter disclosed in the above-referenced patient application Ser. Nos. 09/253,109 and 09/305,613. Accordingly, the second catheter 28 can communicate with the cooling system 14 via coolant supply and return lines 30, 32. Also, the second catheter 28 can communicate with one or more central venous components 34, such as IV infusion devices, drug delivery syringes, blood withdrawal devices, etc. Other central venous components will be understood by those skilled in the art. The component 34 can also be established by a device such as a syringe for administering sedatives, paralytics, neuro-protectants, anti-clot and clot lysis medications. Other medications specific to a particular patient can also be administered through the component 34.

As disclosed in the referenced applications, the second catheter 28 includes a heat exchange region 36 that can be established by one or more membranes such as balloons and hollow fibers. In the case of using hollow fibers, the size and/or the quantity of the fibers would be smaller than those in the first catheter 18.

The second catheter 28 can be advanced into the superior vena cava through the jugular vein or subclavian vein to cool the patient 12 by means of coolant circulating in a closed loop between the cooling system 14 and the heat exchange region 36. In another aspect, the coolant fluid temperature can be adjusted through the controller to provide heated fluid (rather than cooled fluid) to the patient's blood stream whereby the body temperature of the patient increases. In this aspect, the heated fluid is circulated in a closed fluid communication loop between the heat exchanger region 36 and the system 14 to add heat to the patient 12. As mentioned above, the second catheter 28 can also be used to undertake conventional central venous catheter functions.

In one embodiment, the therapeutic systems 10, 11 can include a ventilation system 42 which is connected to the patient 12 via a tube 44 to ventilate the patient. Also, as understood by the present invention, one way to measure ICP is to advance an intra-cranial pressure probe 45, shown schematically in FIGS. 1 and 2, into the head of the patient 12. The pressure probe 45 can include a pressure sensor 45a on the distal end of the probe 45, with the sensor 45a being connected to a pressure indicator 45b that indicates ICP. In this embodiment, the pressure sensor 45a generates a pressure signal that represents the ICP measurement. Additionally, the therapeutic systems 10, 11 can include a cerebral spinal fluid (CSF) drainage system 38 shown in FIGS. 1 and 2. The CSF drainage system is coupled to the patient via a line 40 that communicates with the patient's spine or brain cavity for draining CSF from the patient 12.

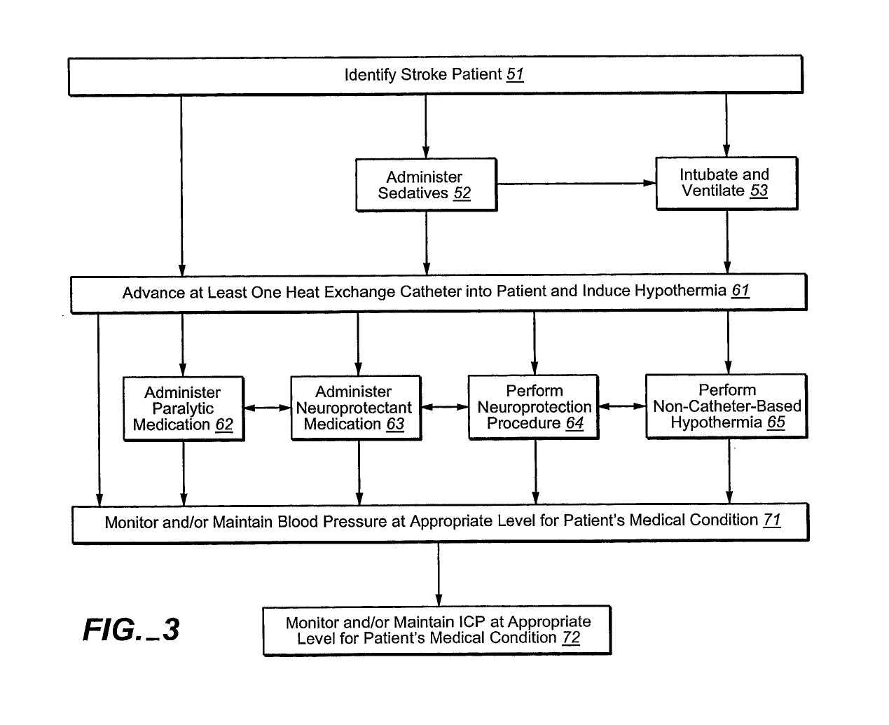

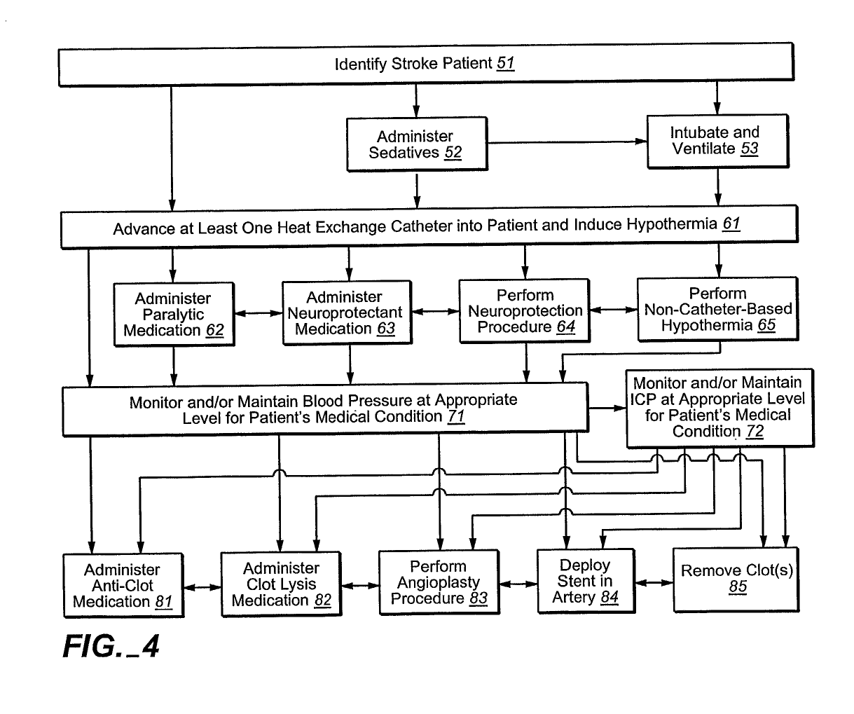

FIG. 3 shows the details of a preferred method for treating a stroke patient 12 who can benefit from hypothermia treatment. In one embodiment, the method for treating a stroke condition includes identifying a stroke patient who can benefit from hypothermia treatment 51. Once the stroke patient 12 is identified, at least one heat exchange catheter 18, 28 is advanced into the patient 12 as shown in block 61. In one embodiment, the heat exchange catheter 18,28 is advanced into the central venous system of the patient 12. Once the heat exchange catheter 18,28 is advanced into the patient 12, hypothermia is induced via the coolant circulation in the heat exchange region 24, 36 of the catheter 18, 28. In another embodiment, the heat exchange catheter 18, 28 is placed through the carotid artery of the patient to lower the temperature of the patient's brain selectively. In yet another embodiment, the patient 12 may receive at least two heat exchange catheters, one placed in the central venous system, and another placed through the carotid artery to lower the body temperature with directed focus on the brain. In this embodiment, the heat exchange rates of each heat exchange catheter may be adjusted separately depending on the patient's relative body and brain temperatures and according to the judgment of the caregiver.

As shown in block 71, the preferred method includes determining the blood pressure and the blood volume of the patient 12, and maintaining the blood pressure and blood volume at the desired levels as determined by the patient's caregiver. In one embodiment, the patient 12 is given a sedative 52. The sedative can be introduced into the patient's blood stream through the medicine dispensing port 25, 37 of the heat exchange catheter 18, 28. In some cases, it may be necessary to intubate and maintain the patient on a ventilator (as represented in block 53) to ventilate the patient 12. Further, the patient 12 may require neuro-protectant medication to prevent further damage to the cerebral tissues. In the present invention, the neuro-protectant medication is introduced into the patient's blood stream through the medicine dispensing port 25, 37 of the heat exchange catheter as represented in block 63. For some patients, a paralytic drug may be needed to curb shivering episodes. Again, as shown in block 62, the paralytic medication maybe given to the patient through the medicine dispensing port 25, 37 of the heat exchange catheter. It will be apparent to those skilled in the art that the medicine dispensing port 25, 37 of the heat exchange catheter can dispense various other medications not presently stated but deemed appropriate for each patient 12 by the caregiver.

For some patients, the present invention includes performing additional neuro-protection procedures 64, such as, but not limited to, bypass surgery and perfusion procedures to deliver oxygenated blood to the brain cells. Additionally, for some patients, the present invention includes at least one non-catheter-based hypothermia treatment 65 being performed on the patient 12. The non-catheter-based hypothermia treatment 65 may include, but is not limited to, applying cooling blankets and/or ice packs on the patient, blowing cold air fans on the patient or immersing the patient in a tub of cold, icy water. Moreover, the present method may include monitoring and maintaining the patient's intracranial pressure (ICP) 72 at a level determined by the caregiver.

FIG. 4 shows the details of a preferred method for treating a ischemic stroke patient 12 who can benefit from hypothermia treatment. In a preferred embodiment directed to the treatment of ischemic stroke patients (shown in FIG. 4), the present invention may include administering anti-clot drugs 81 and or clot lysis drugs 82 can be dispensed through the medicine dispensing port 25, 37 of the heat exchange catheter 18, 28. In another embodiment, the method may include performing an angioplasty procedure on one or more arteries associated with the stroke to eliminate the areas of narrowing in the blood vessels as represented in block 83. In another embodiment, as represented in block 84, a stent is deployed into one or more arteries to provide structural support in the enlarged flow areas of the blood vessels. It will be understood by those skilled in the art which arteries are candidates for angioplasty or stent procedures. In many ischemic stroke patients, the angioplasty and stent procedures are performed on the carotid artery.

Further, in the present invention, any remaining clots to the blood vessels can be cleared away through removing the clot(s) by the application of laser, ultrasonic energies or mechanical device at the blockage areas, or by surgically removing the clots as represented in block 85.

In another aspect, a system for treating a stroke patient includes at least one cooling catheter having a heat exchange region on its distal portion and at least one blood pressure probe to provide an indication of said patient's blood pressure.

As indicated at block 61, hypothermia is induced by advancing the first catheter 18 through the groin into the vena cava, and then circulating coolant through the first catheter 18. Once a target temperature of about 32° C.-36° C. has been reached, the first catheter 18 can be removed and the second catheter 28 advanced into the vena cava through a neck entry point 29 to maintain the target temperature. It is to be understood that while this is one preferred sequence of the order of steps for inducing hypothermia in a stroke patient, other sequences can be used. For example, the first catheter 18 can be used exclusively to the second catheter 28; the second catheter 28 can be used exclusively to the first catheter 18; or both the first and second catheters 18, 28 can be used together simultaneously. Additionally, the caregiver may decide to advance the first catheter 18 into the carotid artery of the patient to cool or maintain the brain temperature.

In the embodiment where ICP is monitored and maintained, the catheters 18, 28 can be left in position once the target temperature is reached. In this manner, if the ICP level of the patient 12 increases and decreasing patient temperature is desired, the cold coolant flow can be re-engaged by simply turning on the coolant circulation flow. The cold coolant flow can be re-engaged manually by a person after observing a change in the patient's ICP level through indicator 45b. Alternatively, through the controller and in its activation of the above-mentioned pump, the coolant circulation flow can be re-engaged automatically by setting an ICP threshold to trigger the start of the coolant circulation flow. Alternatively, if a threshold set point is met white coolant flow is already engaged, the coolant temperature can be changed (decreased or increased) to meet the pre-set medical treatment. In this embodiment, the controller would be electronically connected to the intra-cranial pressure probe 45. It is to be understood that the threshold set point to re-engage the coolant flow can be set for other medical parameters such as, but not limited to, blood pressure, blood volume, etc.

The above method acts are set forth in the presently preferred order, it being understood that the method acts could be performed in other orders as determined to be appropriate. Also, it is to be appreciated that as represented by the arrows shown in FIGS. 3 and 4, the sequences of the method acts can vary and some of the method acts can be omitted and still be within the scope and spirit of the invention. Additionally, the arrows indicate particular sequences of the present invention and without implying that every arrow path must be taken to be within the scope and spirit of the invention.

While the particular METHOD AND SYSTEM FOR TREATING STROKE USING HYPOTHERMIA as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method act in the present disclosure is intended to be dedicated to the public regardless of whether the element, component, or method step is explicitly recited in the claims. No claim element herein is to be construed under the provisions of 35 U.S.C. § 112, sixth paragraph, unless the element is expressly recited using the phrase “means for”.

What is claimed is:

1. A method for treating stroke patients, comprising the acts of:
identifying a stroke patient for treatment;
advancing a heat exchange catheter into said patient; and
inducing hypothermia using said heat exchange catheter.

2. The method of claim 1, further comprising the act of monitoring said patient's blood pressure level.

3. The method of claim 2, further comprising the act of maintaining said patient's blood pressure at a desired blood pressure level.

4. The method of claim 1 wherein said heat exchange catheter is advanced into a central venous vein of said patient.

5. The method of claim 1 wherein said heat exchange catheter is advanced into a carotid artery of said patient.

6. The method of claim 1, further comprising the act of administering a sedative to said patient.

7. The method of claim 6, further comprising the acts of intubating said patient; and then ventilating said patient.

8. The method of claim 1, further comprising the acts of intubating said patient; and then ventilating said patient.

9. The method of claim 1, further comprising the act of administering a paralytic drug to said patient.

10. The method of claim 1, further comprising the act of administering a neuro-protectant drug to said patient.

11. The method of claim 1, further comprising the act of performing a neuro-protection procedure on said patient.

12. The method of claim 1, further comprising the act of performing a non-catheter-based hypothermia procedure on said patient.

13. The method of claim 1, further comprising the act of draining CSF from said patient.

14. The method of claim 13 wherein said draining occurs from a spine cavity.

15. The method of claim 13 wherein said draining occurs from a brain cavity.

16. The method of claim 1, further comprising the act of administering an anti-clot medication to said patient.

17. The method of claim 1, further comprising the act of administering a clot lysis medication to said patient.

18. The method of claim 1, further comprising the act of performing an angioplasty procedure on said patient.

19. The method of claim 1, further comprising the act of deploying a stent in an artery of said patient.

20. The method of claim 1, further comprising the act of removing a clot from an artery of said patient.

21. The method of claim 1, further comprising the act of monitoring said patient's ICP level.

22. The method of claim 21, further comprising the act of maintaining said patient's ICP at a desired ICP level.

23. The method of claim 22, further comprising the act of administering a paralytic drug to said patient.

24. The method of claim 22, further comprising the act of administering a neuro-protectant drug to said patient.

25. The method of claim 22, further comprising the act of performing a neuro-protection procedure on said patient.

26. The method of claim 22, further comprising the act of performing a non-catheter-based hypothermia procedure on said patient.

27. The method of claim 22, further comprising the act of draining CSF from said patient.

28. The method of claim 27 wherein said draining occurs from a spine cavity.

29. The method of claim 27 wherein said draining occurs from a brain cavity.

30. The method of claim 22, further comprising the act of administering an anti-clot medication to said patient.

31. The method of claim 22, further comprising the act of administering a clot lysis medication to said patient.

32. The method of claim 22, further comprising the act of performing an angioplasty procedure on said patient.

33. The method of claim 22, further comprising the act of deploying a stent in an artery of said patient.

34. The method of claim 22, further comprising the act of removing a clot from an artery of said patient.

35. A system for treating a stroke patient, comprising:
at least one cooling catheter having a heat exchange region on a distal portion thereof,
and at least one blood pressure probe to provide an indication of said patient's blood pressure.

36. The system of claim 35, further comprising a drug delivery component and at least one sedative drug in said drug delivery component.

37. The system of claim 35, further comprising a drug delivery component and at least one paralytic drug in said drug delivery component.

38. The system of claim 35, further comprising a drug delivery component and at least one neuro-protectant drug in said drug delivery component.

39. The system of claim 35, further comprising a drug delivery component and at least one anti-clot drug in said drug delivery component.

40. The system of claim 35, further comprising a drug delivery component and at least one clot lysis drug in said drug delivery component.

41. The system of claim 35, further comprising a ventilator for ventilating said patient.

42. The system of claim 35, further comprising a cranial pressure probe advanceable into said patient to provide an indication of pressure level in said patient's head.

43. The system of claim 35, further comprising a cooling system for heat exchange with coolant fluid flowing through said cooling catheter.

44. The system of claim 35, comprising at least two cooling catheters.
